(19) United States
(12) Patent Application Publication
(10) Pub. No.: US 2012/0201908 A1
(43) Pub. Date: Aug. 9, 2012

(54) METHOD OF TREATING BIPOLAR DISORDER OR DEPRESSION USING AN ANTIESTROGEN

(75) Inventor: Kirk Patrick Miller, Superior, CO (US)

(73) Assignee: Lunera Equities, LLLP, Greenwood Village, CO (US)

(21) Appl. No.: 13/390,372

(22) PCT Filed: Aug. 12, 2010
(86) PCT No.: PCT/US2010/045330

§ 371 Date: Apr. 26, 2012

Related U.S. Application Data
(60) Provisional application No. 61/235,090, filed on Aug. 19, 2009.

(51) Int. Cl. A61K 31/135; A61K 31/4196; A61P 25/24; A61K 31/437; A61K 33/00; A61K 31/5513; A61K 31/4535; A61K 31/56

(52) U.S. Cl. 424/722; 514/648; 514/324; 514/383; 514/177; 514/178; 514/300; 514/221

(57) ABSTRACT

The disclosure provides methods of treating bipolar disorder by administering an antiestrogen to reduce the severity and frequency of mood episodes. The disclosure further provides methods of treating depression with an antiestrogen in a descending dose protocol.

This application is being filed on 12 Aug. 2010, as a PCT International Patent application in the name of Lunera Research, Inc., a U.S. national corporation, applicant for the designation of all countries except the U.S., and Kirk Patrick Miller, a citizen of the U.S., applicant for the designation of the U.S. only, and claims priority to U.S. Provisional Patent Application Ser. No. 61/235,090 filed on 19 Aug. 2009.

### FIELD OF THE DISCLOSURE

The disclosure provides methods of treating bipolar disorder by administering an antiestrogen to reduce the severity and frequency of mood episodes. The disclosure further provides methods of treating depression with an antiestrogen in a descending dose protocol.

### BACKGROUND OF THE DISCLOSURE

Depression, also known as clinical depression, major depression, unipolar depression, major depressive disorder and recurrent depressive disorder, is a common, debilitating disorder. The lifetime prevalence rate of major depression in the U.S. is about 16% in the total adult population. (Marcotte et al., Prevalance and patterns of major depressive disorder in the United States labor force, J. Mental Health Policy Econ. 2,123-131, 1999). There are several types of depressive disorders. See, for example, National Institutes of Mental Health, Depression, NIH Publication No. 08-3561, revised 2008. The most common depressive disorders are major depression and dysthymic disorder.

Major depressive disorder, or major depression, is characterized by a combination of symptoms that interfere with the ability to work, sleep, study, eat and enjoy once-pleasurable activities. Major depression is disabling and interferes with normal life functioning. An episode of major depression may only occur once, or may recur throughout a person's life. Major depressive disorder (MDD) is a common disorder, widely distributed in the population, and usually associated with substantial symptom severity and role impairment. In a survey of adults in the 48 contiguous United States, mean episode duration of MDD was 16 weeks. (Kessler et al., JAMA 2003, 289:3095-3105).

Dysthmic disorder, also called dysthymia, is characterized by longer term episode duration, but less severe symptoms than MDD, over a period of two years or greater. Symptoms may not disable a person, but can prevent one from functioning normally or feeling well. People with dysthymia may also experience one or more episodes of major depression. Other forms of depression include psychotic depression, postpartum depression and seasonal affective disorder (SAD). Depression often coexists with other illnesses including anxiety disorders such as post-traumatic stress disorder (PTSD), obsessive-compulsive disorder, panic disorder, social phobia and generalized anxiety disorder. Alcohol abuse, other substance abuse, heart disease stroke, cancer, HIV/AIDS, diabetes, and Parkinson's disease often co-occur with depression. There is increasing evidence that treating the depression can also help improve the outcome of treating the co-occurring illness. (Katon and Ciechanowski, Impact of major depression on chronic medical illness. J. Psychosomatic. Res., 2002, 53:859-863).

Diagnosis of depression is made by a psychiatrist, psychologist or general practitioner who assesses the person's current circumstances, biographical history, family medical history, and current symptoms and discusses the person's alcohol and drug use. The assessment also includes a mental state examination which is an assessment of the person's current mood, and thought content, and the presence of themes of hopelessness or pessimism, self-harm or suicide and an absence of positive thoughts or plans. Before diagnosing a major depressive disorder, a doctor usually performs a medical examination to rule out other causes of symptoms. These include blood tests such as TSH and thyroxine to exclude hypothyroidism, basic electrolytes and serum calcium to rule out a metabolic disturbance, and a full blood count to rule out systemic infection or chronic disease. Testosterone levels may be evaluated to diagnose hypogonadism, a cause of depression in men. Cognitive testing and brain imaging can help distinguish depression from dementia in depressed older people. The most widely used criteria are found in the American Psychiatric Association's revised fourth edition of the Diagnostic and Statistical Manual of Mental Disorders (DSM-IV-TR), 2000 and in the World Health Organization's International Statistical Classification of Diseases and Related Health Problems (ICD-10) which uses the name recurrent depressive disorder. Major depressive disorder is classified as a mood disorder in DSM-IV-TR. A major depressive episode is characterized by the presence of a severely depressed mood that persists for at least two weeks. Episodes may be isolated or recurrent and are categorized as mild, moderate or severe. If a patient has had an episode of mania or marked elevated mood a diagnosis of bipolar disorder is made instead.

Symptoms of depression include persistent sad, anxious or “empty” feelings. Feelings of hopelessness or pessimism, feelings of guilt, worthlessness or helplessness, loss of interest in activities once thought pleasurable, including sex, fatigue and decreased energy, difficulty concentrating, remembering details and making decisions, insomnia, early morning wakefulness, or excessive sleeping, overeating, or appetite loss, thoughts of suicide or suicide attempts, persistent aches or pains, headaches, and cramps or digestive problems that do not ease with treatment.

The causes of depression are not fully known. There is likely a combination of various genetic, biologic, and environmental factors at work. McKinlay et al., The relative contributions of endocrine changes and social circumstances to depression in mid-aged women, J. Health Social Behav., 1987, 28:345-363.

The most common treatments of depression are psychotherapy, medication and electroconvulsive therapy (ECT). Psychotherapy is particularly preferred in the treatment of young people under 18 years. ECT is used as a last resort.

Antidepressant medications often work to normalize certain naturally occurring brain neurotransmitters such as serotonin, norepinephrine or dopamine. Selective serotonin reuptake inhibitors (SSRIs) are a newer, popular class of antidepressants that includes fluoxetine (Prozac®), citalopram (Celexa®), sertraline (Zoloft®) and several others. Serotonin and norepinephrine reuptake inhibitors (SNRIs) include venlafaxine (Effexor®) and duloxetine (Cymbalta®). SSRIs and SNRIs are more popular than the older classes of antidepressants such as the tricyclics, e.g. imipramine (Tofranil®), and the monoamine oxidase inhibitors (MAOIs), because they tend to have fewer side effects. Sometimes an anticonvulsant such as clonazepam (Klonopin®) is used in combination with another drug such as an SSRI in the treatment of, for example, clinical depression. However, medications affect everyone differently. To find the most effective antidepressant with few or the most tolerable side effects, the dosages must be adjusted, and if necessary, various combinations of different classes of antidepressants can be tried. Response can take at least six to eight weeks from the start of treatment to remission. Further, medication is typically continued for 16-20 weeks after remission to minimize the chance of recurrence. People with chronic depression may take medication indefinitely to avoid relapse. Common side effects of antidepressants include headache, nausea, sleep problems, agitation, and sexual problems such as reduced sex drive. An FDA warning says patients of all ages should be watched closely especially during the first few weeks of treatment. Antidepressants were found to increase risk compared to placebo of suicidal thinking and behavior (suicidality) in children, adolescents, and young adults in short-term studies of major depressive disorder and other psychiatric disorders. Other possible side effects are depression that gets worse, unusual changes in behavior such as trouble sleeping, agitation, or withdrawal from normal social situations.

Clearly, due to variation of individual response to known classes of antidepressants, as well as certain unacceptable side effects, alternative methods of treating various depressive disorders are desirable.

Bipolar disorder, also known as manic depression, manic depressive disorder or bipolar affective disorder, is a brain disorder that causes unusual shifts in mood, energy, activity levels and the ability to carry out daily tasks. Bipolar disorder is not as common as major depression or dysthymia. Bipolar disorder involves cycling mood changes between periods of excitability (mania) alternating with periods of depression. The “mood swings” can be very abrupt between mania and depression. Symptoms can be severe, interfering with day to day tasks, and resulting in damaged relationships, poor job or school performance, and even suicide. See, for example, National Institutes of Mental Health, Bipolar Disorder, NIH Publication No. 08-3679, revised 2008. Bipolar disorder affects about 5.7 million Americans, or about 2.6 percent of the U.S. population age 18 and older in a given year. (Kessler et al., Prevalence, severity, and comorbidity of 12-month DSM-IV disorders in the national comorbidity survey replication, Arch. Gen. Psychiatry; 2005, 62:617-709).

Symptoms of bipolar disorder include unusually intense emotional states called “mood episodes”. An overly joyful or overexcited state is called a manic episode; an extremely sad or hopeless state is called a depressive episode. Sometimes, a mood episode includes symptoms of both mania and depression. This is called a mixed state. People with bipolar disorder may also be explosive and irritable during a mood episode. A person may be having an episode of bipolar disorder if he or she has a number of manic or depressive symptoms for most of the day, nearly every day for a period of one or two weeks.

Symptoms of mania or a manic episode include mood changes such as an elevated mood, a long period of feeling “high”, or an overly happy or outgoing mood, increased energy, hyperactivity, an extremely irritable mood, agitation, feeling “jumpy” or “wired”, and little need for sleep. Behavioral changes include talking very fast, jumping from one idea to another, having racing thoughts, being easily distracted, increasing goal-directed activities such as taking on new projects, over-involvement in activities, being restless, sleeping little, having an unrealistic belief in one's abilities, lack of self control, poor temper control, behaving impulsively and increased high-risk behaviors such as binge eating, drinking and/or drug use, impaired judgment, spending sprees, sexual promiscuity, and impulsive business investments.

Symptoms of depression or a depressive episode include mood changes such as a long period of feeling worried or empty, withdrawal from activities once enjoyed, such as sex, feeling tired or “slowed down”, feelings of worthlessness, hopelessness, or guilt, loss of self esteem, persistent sadness, withdrawal from friends, difficulty concentrating, remembering and making decisions, being restless or irritable, fatigue or listlessness, eating disturbances such as loss of appetite and weight loss or overeating and weight gain, sleep disturbances such as excessive sleepiness or the inability to sleep, persistent thoughts of death, suicidal thoughts, or attempting suicide.

In addition to mania and depression, bipolar disorder can cause a range of moods from severe depression to moderate depression to mild low mood through to a normal or balanced mood to hypomania to severe mania. During a mixed state, symptoms include agitation, trouble sleeping, major changes in appetite, and suicidal thinking. People in a mixed state may feel very sad or hopeless while feeling extremely energized. Sometimes severe episodes of mania or depression are accompanied by psychotic symptoms such as hallucinations or delusions which tend to reflect the person's extreme mood. As a result, people with bipolar disorder who have psychotic symptoms are sometimes misdiagnosed as having schizophrenia, another severe mental illness associated with hallucinations and delusions. People with bipolar disorder also have behavioral problems, such as alcohol abuse or substance abuse.

Bipolar disorder often develops in a person's teen or early adult years. About half of all cases start before age 25. Some people have their first symptoms during childhood, while others may develop symptoms late in life. Bipolar disorder usually lasts a lifetime. Episodes of mania and depression typically come back over time. Between episodes, many people are free of symptoms, but some people have lingering symptoms.

According to the Diagnostic and Statistical Manual of Mental Disorders, or DSM, there are four basic types of bipolar disorder: Bipolar I disorder is mainly defined by manic or mixed episodes that last at least seven days, or by manic symptoms so severe that the person requires immediate hospital care. Usually the person also has depressive episodes, typically lasting at least two weeks. The symptoms of mania or depression must be a major change from the person's normal behavior. Bipolar II Disorder is defined by a pattern of depressive episodes shifting back and forth with hypomanic episodes, but no full-blown manic or mixed episodes. Bipolar Disorder Not Otherwise Specified (BP-NOS) is diagnosed when a person has symptoms that do not meet the criteria for either bipolar I or II. The symptoms may not last long enough, or the person may have too few symptoms to be diagnosed with bipolar I or II. However, the symptoms are clearly out of the person's normal range of behavior. Cyclothymic Disorder, or Cyclothymia, is a mild form of bipolar disorder. People with cyclothymia have episodes of hypomania that shift back and forth with symptoms of mild depression for at least two years. However, the symptoms do not meet the diagnostic requirements for any other type of bipolar disorder. Some people are diagnosed with rapid-cycling bipolar disorder when a person has had four or more episodes of major depression, mania, hypomania or mixed symptoms within a year. Some people experience more than one episode in a week, or even within one day.

Bipolar disorder tends to get worse if not treated; over time both the frequency and severity of symptoms can increase. In most cases, treatment can help reduce the frequency and severity of episodes.

Diagnosis of bipolar disorder is made by a doctor, for example a psychiatrist, or mental health professional by conducting a complete diagnostic evaluation which includes any family history of bipolar disorder or other mental illness and a complete history of symptoms. The doctor or mental health professional will also typically talk to a person's close relatives or spouse to note how they describe the person's symptoms and family medical history.

There is currently no cure for bipolar disorder, but several treatment options exist. Proper treatment can help most people gain better control of their mood swings and related symptoms. Not everyone responds to medication in the same way. Several different medications may need to be tried before the best course of treatment is found. The subject is asked to keep a chart of daily mood symptoms, treatments, sleep patterns, and life events, sometimes called a “daily life chart”, or “mood chart”; this helps the doctor track and treat the illness most effectively. If symptoms change or side effects become serious, the doctor may switch or add medications.

Mood stabilizing medications are usually the first choice to treat bipolar disorder. Generally, people with bipolar disorder continue treatment with mood stabilizers for years. The term “mood stabilizer” is used to define a treatment that decreases the vulnerability to subsequent episodes of mania or depression and that does not exacerbate the current acute episode when administered during the acute, continuation, or maintenance phase of treatment. See generally, Sachs, Bipolar mood disorder: practical strategies for acute and maintenance phase treatment, J. Clin. Psychopharmacol., 1996, 16(2), Suppl. 1, 32S-47S.

The beneficial effects of mood stabilizers are known to require a lag period for onset of action and are generally not immediately reversed upon drug discontinuation; such patterns of effects suggest alterations at the genomic level. A “mood stabilizer” is a medication used to treat mood disorders characterized by intense and sustained mood shifts. Except for lithium, these medications are typically anticonvulsants. Anticonvulsant drugs are used to treat seizures, but can also be used to help control moods.

Typically, multiple medications are utilized in treatment of a bipolar disorder. For example, Lithium and/or an anticonvulsant can be used and often combined with an antidepressant or antipsychotic. Often several drugs are necessary; however, it is difficult to predict the effect of various drug combinations in different individuals. During the course of treatment, certain drugs may be discontinued due to unpleasant or dangerous side effects. When discontinuing a drug, particularly an antidepressant, or an antipsychotic, the patient must be carefully monitored for withdrawal symptoms or re-emergence of symptoms, e.g., depression. To avoid or minimize withdrawal symptoms, the dosage of a drug is decreased over time prior to discontinuation when changing a drug protocol.

Lithium (Eskalith®, or Lithobid®) was the first mood-stabilizing medication approved by the FDA for the treatment of mania. It can be very effective in controlling symptoms of mania and preventing recurrence of manic and depressive episodes. Common side effects include mild hand tremor; mild thirst; temporary mild nausea and general discomfort at the beginning of treatment. Severe side effects include severe allergic reaction; blurred vision; confusion; diarrhea; drowsiness; excessive weight gain; fainting; giddiness; inability to control bladder or bowels; increased thirst; involuntary twitching or muscle movements; muscle weakness; persistent headache; severe nausea; slurred speech; slow or irregular heartbeat; swelling of ankles or wrists; and vomiting.

Valproic acid, or divalproex sodium (Depakote®), approved by the FDA in 1995 for treating mania, is a popular alternative to lithium for treating bipolar disorder. It is generally as effective as lithium. Common side effects include abdominal or stomach cramps; change in menstrual periods; diarrhea; hair loss; indigestion; loss of appetite; nausea and vomiting; trembling of hands and arms; and unusual weight gain or loss. Less common side effects include clumsiness or unsteadiness; dizziness; drowsiness; headache; constipation; skin rash; unusual excitement; restlessness; or irritability.

Lamotrigine (Lamictal®) is an anticonvulsant drug, first introduced in the 1990's for the treatment of partial seizures, that has recently gained approval for maintenance treatment of bipolar disorder. While traditional anticonvulsant drugs are predominantly antimanics, lamotrigine is most effective in the treatment and prophylaxis of bipolar depression. However, lamotrigine is not approved on label for treatment of acute bipolar symptoms. The dosage must be increased slowly from sub-therapeutic level to therapeutic level. Lamotrigine prescribing information has a black box warning about life-threatening skin conditions, including Stevens-Johnson Syndrome and Toxic Epidermal Necrolysis. The manufacturer states that nearly all cases start within the first 2-8 weeks. Common side effects include headaches, dizziness and insomnia. About 5-10% of patients will develop a rash; about one in 1,000 will develop a serious rash. Other side effects include acne, skin irritation, vivid dreams, night sweats, muscle aches, weight changes, changes in libido, hair loss, frequent urination, and nausea.

Other anticonvulsant medications including gabapentin (Neurontin®), topiramate (Topamax®), and oxcarbazepine (Trileptal®), clonazepam (Klonopin®) are also sometimes prescribed; although no large studies have shown these medications are more effective than mood stabilizers.

Valproic acid, lamotrigine, and other anticonvulsant medications have an FDA warning that states that use may increase the risk of suicidal thoughts or behaviors. People taking these medications should not make any changes without talking to their health care professional. People taking anticonvulsant medications for bipolar or other illnesses should be closely monitored for new or worsening symptoms of depression, suicidal thoughts or behavior, or any unusual changes in mood or behavior.

In an attempt to determine the mechanism of action for mood stabilizers, Manji et al. found that administration of two structurally dissimilar agents, lithium and valproate, bring about a similar reduction in protein kinase C alpha and epsilon isozymes in rat frontal cortex and hippocampus. (Manji et al., Modulation of CNS signal transduction pathways and gene expression by mood-stabilizing agents: therapeutic implications, J. Clin. Psychiatry, 1999; 60 Suppl. 2:27-39.)

Atypical antipsychotic medications are also sometimes used to treat symptoms of bipolar disorder. Olanzapine (Zyprexa®) when given with an antidepressant medication may help relieve symptoms of severe mania or psychosis. Aripiprazole (Abilify®) is approved for treatment of a manic or mixed episode. Aripiprazole is also sometimes used for maintenance treatment after a severe or sudden episode. Quetiapine (Seroquel®) relieves the symptoms of severe and sudden manic episodes. Risperidone (Risperdal®) and ziprasidone (Geodon®) are other atypical antipsychotics sometimes used for controlling manic or mixed episodes. Common side effects of atypical antipsychotics include drowsiness, dizziness, blurred vision, rapid heartbeat, skin rashes and menstrual problems for women. In rare cases, long term use of atypical antipsychotics can result in tardive dyskinesia, which causes uncontrolled muscle movements.

Antidepressant medications are sometimes used to treat or prevent symptoms of depression in bipolar disorder, in combination with a mood stabilizer. Taking only an antidepressant, however, has been noted to increase the risk of switching to mania or hypomania, or of developing rapid cycling symptoms. (Thase et al., Biol. Psychiatry 2000; 48(6):558-572.) Fluoxetine (Prozac®), Paroxetine (Paxil®), sertraline (Zoloft®), and bupropion (Wellbutrin®) are examples of antidepressants that may be prescribed to treat symptoms of bipolar depression. A recent large scale study showed that for many people, adding an antidepressant to a mood stabilizer is no more effective in treating depression than using only a mood stabilizer. (Sachs et al., N. Engl. J. Med. 2007 Apr. 26; 356(17):1711-1722). Common side effects of antidepressants include headache, nausea, sleep problems, agitation, and sexual problems such as reduced sex drive. An FDA warning says patients of all ages should be watched closely especially during the first few weeks of treatment. Possible side effects are depression that gets worse, suicidal thinking or behavior, or unusual changes in behavior such as trouble sleeping, agitation, or withdrawal from normal social situations.

If a person with bipolar disorder develops any severe side effects from a medication, he or she is advised to speak to the prescribing physician as soon as possible. People being treated for bipolar disorder should not stop taking a medication without talking to a doctor first. Suddenly stopping a medication may lead to rebound or worsening of bipolar disorder symptoms.

Alternatives to prescription medications are sometimes utilized in the treatment of bipolar disorder. Sometimes psychotherapy is used in addition to medication to treat bipolar disorder. Another treatment is Electroconvulsive Therapy (ECT). Although highly effective for severely depressive, manic, or mixed episodes, ECT is not generally a first line treatment. The herbal supplement, St. John's wort (Hypericum perforatum), is marketed as a natural antidepressant and can cause a switch to mania in some people with bipolar disorder. St. John's wort can also make other medications less effective including antidepressant and anticonvulsant medications.

Clearly, due to variation of individual response to classic mood stabilizers, and anticonvulsants, as well as certain unacceptable side effects; alternative methods of treating bipolar disorder are desirable. Further, it would be desirable to treat a patient diagnosed with a bipolar disorder effectively with a single drug while achieving or maintaining mood stabilization in the patient.

### SUMMARY OF THE DISCLOSURE

In one embodiment, the disclosure provides a method of treating a bipolar disorder, comprising administering an antiestrogen, or a pharmaceutically acceptable salt thereof, in an amount effective to alleviate or prevent mood swings in a subject in need thereof. In certain aspects, the bipolar disorder is selected from the group consisting of bipolar disorder I, bipolar disorder II, cyclothymic disorder, and bipolar disorder not otherwise specified.

In one embodiment, the method of treating bipolar disorder with a pharmaceutically active antiestrogen involves administration of, for example, a pharmaceutically effective amount of antiestrogen selected from tamoxifen, raloxifene, anastrozole, letrozole, exemestane, vorozole, formestane, and fadrozole, or a pharmaceutically acceptable salt thereof, in an amount effective to alleviate or prevent one or more symptoms of bipolar disorder selected from the group consisting of mood swings and mood episodes.

In one aspect, the antiestrogen is tamoxifen citrate. In a specific aspect, the tamoxifen citrate is administered in the amount of about 10 to about 20 mg every day or every other day. In another aspect, the antiestrogen is anastrozole. In a specific aspect, the anastrozole is administered in the amount of about 0.25 to about 1 mg every day or every other day. In another aspect, the method further comprises administering a pharmaceutically effective dose of at least one member of the group consisting of lithium, a pharmaceutical antidepressant, an anticonvulsant, a mood stabilizer, an antipsychotic agent and a benzodiazepine.

In a further embodiment, the disclosure provides a method of treating a bipolar disorder, comprising administering an antiestrogen, or a pharmaceutically acceptable salt, ester, or prodrug thereof, in an amount sufficient to reduce one or more of the severity of mood episodes and the frequency of mood episodes in a subject in need thereof. In one aspect, the mood episodes comprise symptoms of depression. In certain specific aspects, the antiestrogen is selected from tamoxifen, anastrozole, or a pharmaceutically acceptable salt thereof.

In another embodiment, the disclosure provides for use of an antiestrogen, or a pharmaceutically acceptable salt thereof, in the manufacture of a composition for treating a bipolar disorder in a patient in need thereof; wherein the composition is to be administered in an amount effective to alleviate or prevent one or more symptoms of the bipolar disorder selected from the group consisting of mood swings and mood episodes.

In another embodiment, the disclosure provides a method of treating a bipolar disorder with an antiestrogen further comprising the steps of observing stabilized behavior in the patient; and decreasing the dose or discontinuing the administration of one or more other concurrently administered drugs selected from the group consisting of lithium, a pharmaceutical antidepressant, an anticonvulsant, a mood stabilizer, an antipsychotic agent and a benzodiazepine that is being concurrently administered to the patient.

In a further embodiment, the disclosure provides a method of treating a clinical depression comprising administering an effective amount of an antiestrogen, or a pharmaceutically acceptable salt thereof, to treat depression in a patient in need thereof. In one aspect, the depression is selected from unipolar depression, major depressive disorder, psychotic depression, dysthymia, bipolar depression, treatment-resistant depression, single episodic or recurrent major depressive disorders. In a particular aspect, the bipolar depression is in an individual suffering from bipolar disorder I, bipolar disorder II, or cyclothymic disorder.

In one embodiment, the method of treating depression with an antiestrogen involves administration of a pharmaceutically active antiestrogen. In one aspect, the antiestrogen is selected from tamoxifen, raloxifene, anastrozole, letrozole, exemestane, vorozole, formestane, and fadrozole, or a pharmaceutically acceptable salt thereof. In one specific aspect, the antiestrogen is tamoxifen citrate. In a specific aspect, the tamoxifen citrate is administered in a daily descending dosing schedule. For example, one specific method of treating depression comprises administering tamoxifen citrate in a daily descending dosing schedule comprising about 20 mg on day one, about 10 mg on day two, about 5 mg on day three, about 2.5 mg on day four and about 2.5 mg on day five.

In another specific aspect, the method of treating depression with an antiestrogen involves administration of the antiestrogen anastrozole. In a specific aspect, the anastrozole is administered in a daily descending dosing schedule. For example, in one specific aspect, the method of treating depression comprises administering anastrozole in a daily descending dosing schedule comprising administering about 1 mg on day one, about 0.5 mg on day two, about 0.25 mg on day three and about 0.25 mg on day four, and optionally further comprising administering 0.25 mg of anastrozole on day six and 0.25 mg on day eight.

In one aspect, the method comprises administering an antiestrogen at the first sign of acute severe depression. In a further aspect, the method further comprises administering a pharmaceutically effective dose of at least one member of the group consisting of lithium, a pharmaceutical antidepressant, an anticonvulsant, a mood stabilizer, an antipsychotic agent and a benzodiazepine.

### BRIEF DESCRIPTION OF THE DRAWINGS

FIG. 1 shows the drug protocol for Subject D during the study.

FIG. 2 shows the drug protocol for Subject E during the study.

FIG. 3 shows the drug protocol for Subject F during the study.

### DETAILED DESCRIPTION OF THE DISCLOSURE

In one embodiment, the disclosure provides methods of treating depression by administering an antiestrogen. In one aspect, the method employs a daily descending dose schedule of administering an antiestrogen to a subject exhibiting signs of depression. It has been surprisingly found that treatment of the symptoms of depression with an antiestrogen will result in a relatively rapid decrease in the severity of symptoms, with noticeable relief within about one to two days of administration of the first dose. In a specific aspect, the depression is acute severe depression. In another aspect, the depression is an episode of depression in a subject diagnosed with major depression, unipolar depression, dysthymia, bipolar depression, treatment-resistant depression, bipolar disorder I, bipolar disorder II, and cyclothymic disorder. The depression may occur in either single episodic and/or recurrent major depressive disorders. In a further aspect, the method of treating depression further comprises administering a pharmaceutically effective dose of at least one member of the group consisting of lithium, a pharmaceutical antidepressant, an anticonvulsant, a mood stabilizer, an antipsychotic agent and a benzodiazepine.

In another embodiment, the disclosure provides methods of treating bipolar disorder with an antiestrogen to alleviate or prevent mood swings. In a further embodiment, the disclosure provides a method of treating a bipolar disorder to reduce or prevent the severity of mood episodes and the frequency of mood episodes. The bipolar disorder to be treated is selected from the group consisting of bipolar disorder I, bipolar disorder II, cyclothymic disorder, and bipolar disorder not otherwise specified. In a further aspect, the method of treating bipolar disorder further comprises administering a pharmaceutically effective dose of at least one member of the group consisting of lithium, a pharmaceutical antidepressant, an anticonvulsant, a mood stabilizer, an antipsychotic agent and a benzodiazepine.

In one aspect of the disclosure, a bipolar mood chart is used to track patient information through time for variables such as mood, medication, sleep and life events. The mood chart can be used to discern the effect of various events, types of medication, dosage of medication, and combination of medications that may be more or less effective to treat various symptoms over time. Any mood chart may be utilized to track patient information. The mood chart may be selected from, for example, the NIMH Daily Mood Chart; Mood Chart from the Massachusetts General Hospital Bipolar Clinic and Research program, also known as a Gary Sachs mood chart; or Black Dog Institute mood chart. Alternatively, an online mood chart program can be utilized, such as moodtracker.com. In one aspect, the type of mood chart, or modification thereof, is selected by the patient's physician. In another aspect, the mood chart is a Gary Sachs mood chart, or modification thereof. Sachs, Bipolar mood disorder: practical strategies for acute and maintenance phase treatment, J. Clin. Psychopharmacol., 1996, 16(2), Suppl. 1, 32S-47S; p. 47S. In another aspect, the patient completes the mood chart, optimally, on a regular basis, such as one time per day, twice per day, every other day, etc. In a preferred aspect, the patient fills out the mood chart on a regular basis and a family member, friend, roommate or clinical or institutional staff member who interacts with the patient on a regular basis, also records a parallel mood chart on a regular basis. Optimally, the family member or friend lives with, or interacts with the patient on a routine basis. In one aspect, the mood chart is used to compare daily treatment with mood, for example, depressed or elevated mood and other such factors as menses for women, sleep duration and levels of anxiety and irritability, or other symptoms. In another aspect, the mood chart is completed on a regular basis by a family member, friend, roommate or clinical or institutional staff member who interacts with the patient on a regular basis.

The term “subject”, or “patient”, refers to an animal, for example a mammal, such as a human, who is the object of treatment. In a specific aspect, the patient, or subject, is diagnosed as suffering from a bipolar disorder or a clinical depression. The subject, or patient, may be either male or female.

As used herein, the term “about” when referring to a number of days includes the specified number of days±2 days.

The term “about” when used to refer to a therapeutically effective amount of an antiestrogen includes the specified amount±10%.

The term “bid” means administered twice a day.

The term “hs” refers to at bedtime.

The term “qd” refers to administered once every day.

The term “po” refers to administered by mouth.

The term “qh” refers to every hour.

The term “prn” refers to administered as needed.

The term “qam” refers to every morning.

The term “qhs” refers to every bedtime.

The term “qod” refers to every other day.

The term “antiestrogen”, as used herein, is used to define a pharmaceutically effective substance that prevents cells from making or using estrogen. The antiestrogen of the disclosure is selected from, for example, tamoxifen, raloxifene, anastrozole, letrozole, exemestane, vorozole, formestane, and fadrozole, or pharmaceutically acceptable salts thereof. In one specific aspect, the antiestrogen is tamoxifen citrate. In another specific aspect, the antiestrogen is anastrozole. The term antiestrogen also includes active metabolites of known antiestrogens, for example, tamoxifen, raloxifene, anastrozole, letrozole, exemestane, vorozole, formestane, and fadrozole, or pharmaceutically acceptable salts thereof.

Certain antiestrogens may stop some cancer cells from growing and are used to prevent and treat certain types of cancer, for example, breast cancer. In premenopausal women, most estrogen is produced in the ovaries; in post-menopausal women, most estrogen is produced in the adrenal gland from conversion of androgens.

Tamoxifen is one example of an antiestrogen. Tamoxifen, in the form of the pharmaceutically acceptable salt tamoxifen citrate, or (Z)2-[4-(1,2-diphenyl-1-butenyl)phenoxy]-N,N-dimethylethanamine 2-hydroxy-1,2,3-propanetricarboxylate (1:1), was originally approved by the FDA in 1977 as Nolvadex® (AstraZeneca) to treat breast cancer. Tamoxifen citrate is now available as a generic drug and is used to treat early and advanced estrogen receptor positive breast cancer in pre- and post-menopausal women. Tamoxifen citrate is typically administered as an oral tablet in 10 mg and 20 mg dosage forms. The tamoxifen 10 mg tablets contain 10 mg of tamoxifen base as 15.2 mg of tamoxifen citrate; and the 20 mg tablets contain 20 mg of tamoxifen base as 30.4 mg of tamoxifen citrate. Tamoxifen is the most common treatment for male breast cancer. It is also approved for the prevention of breast cancer in high risk women. In breast cancer treatment, tamoxifen can act as a prodrug which is oxidatively metabolized in the liver by certain cytochrome P450 enzymes to active metabolites such as 4-hydroxytamoxifen and N-desmethyl-4-hydroxytamoxifen; these active metabolites compete with estrogen for binding to the estrogen receptor in breast tissue. Tamoxifen treatment interferes with the cell-division cycle by causing breast cancer cells to remain in the G₀ and G₁ phases of the cell cycle, and therefore blocks cell division.

Tamoxifen is also known as a selective estrogen receptor modulator (SERM), since it does not act as an estrogen receptor antagonist in all types of tissue. One beneficial side effect of tamoxifen is that it acts to prevent bone loss by acting as an estrogen receptor agonist in bone cells. By inhibiting osteoclasts, tamoxifen helps prevent osteoporosis. (Krum et al., 2008, EMBO J.; 27(3):535-545).

Tamoxifen has also been shown to be effective in treatment of mania in patients with bipolar disorder, purportedly by blocking protein kinase C (PKC), an enzyme that regulates neuron activity in the brain. Researchers believe that PKC is over-active during the mania phase in bipolar patients. (Yildiz et al., Arch. Gen. Psychiatry, 2008; 65(3):255-263).

Tamoxifen citrate has been sold as Nolvadex® and is currently sold as an oral tablet in 10 or 20 mg base equivalent per tablet by a variety of pharmaceutical companies including Aegis Pharmaceuticals, Inc.; Barr; Mylan; Teva; and Watson Labs.

Active metabolites of tamoxifen include, for example, 4-hydroxytamoxifen and 4-hydroxy-N-desmethyltamoxifen. (Borgna et al., 1981, Hydroxylated metabolites of tamoxifen are formed in vivo and bound to estrogen receptor targets, J. Biol. Chem., 256(2):859-868; Stearns et al 2003, Active tamoxifen metabolite plasma concentrations after coadministration of tamoxifen and the selective serotonin reuptake inhibitor paroxetine, J. Nat'l. Cancer Inst, 95(23): 1758-64).

Another example of an antiestrogen for the purposes of the disclosure is raloxifene, which is used to protect against bone loss (osteoporosis) in post-menopausal women. Raloxifene hydrochloride (Evista®, Lilly) is considered to be an estrogen agonist/antagonist and is sold as an 60 mg oral tablet. Evista is approved for inhibiting bone resorption, treatment or prevention of osteoporosis, prevention of breast cancer, and prevention and treatment of osteoporosis in postmenopausal women. Raloxifene is disclosed in U.S. Pat. Nos. 6,458,811; 6,797,719; and 6,894,064, each of which is incorporated herein by reference.

Aromatase inhibitors (AIs) are a class of antiestrogen that blocks the conversion of androgens to estrogen; this lowers the estrogen level, and slows the growth of certain cancer cells which rely upon estrogen for growth. Aromatase inhibitors are used to treat breast cancer and ovarian cancer in post-menopausal women. Aromatase inhibitors have also been shown to reverse age-related declines in serum testosterone. (Leder et al., J. Clin. Endocrinol. Metab., 2004, 89(3):1174-1180).

Aromatase inhibitors are categorized into two types. Type I AIs include irreversible steroidal inhibitors such as exemestane (Aromasin®), which form a permanent covalent bond with the aromatase complex. Exemestane is sold as a 25 mg oral tablet by Pharmacia and Upjohn. Exemestane is approved for the treatment of advanced hormone-dependent breast cancer. An active metabolite of exemestane is 17-hydroxyexemestane. (Hutson et al., 2005, Effect of exemestane on tamoxifen pharmacokinetics in postmenopausal women treated for breast cancer., Clin. Cancer Res. 2005; 11(24) 8722-8727.)

Type II AIs include non-steroidal inhibitors such as anastrozole (Arimidex®, AstraZeneca) and letrozole (Femara®, Novartis) which inhibit the enzyme by reversible competition. Anastrozole was originally approved to treat breast cancer in 1995 as Arimidex® (AstraZeneca) in 1 mg oral tablets, and is now available as a generic drug from several manufacturers including, for example, Teva, Synthon, Roxane, Zydus, Stason, Watson, Sandoz, Natco, Fresenius Kabi, Accord, Dr. Reddy's and Three Rivers Phams. Arimidex is approved for adjuvant treatment of post-menopausal women with hormone receptor-positive or hormone receptor unknown locally advanced or metastatic breast cancer. In cancer treatment, the dose of anastrozole is one 1 mg oral tablet per day. Anastrozole is disclosed in U.S. Pat. No. RE36617, which is incorporated herein by reference. Letrozole is sold as a 2.5 mg oral tablet by Mylan as Letrozole or by Novartis Pharmaceuticals as Femara®. Letrozole is approved for the treatment of advanced breast cancer in postmenopausal women with disease progression following antiestrogen therapy. Letrozole is disclosed in U.S. Pat. No. 4,978,672, which is incorporated herein by reference. Other selective aromatase inhibitors include vorozole (Rivizor®), formestane (Lentaron®; intramuscular depot injectable), and fadrozole (Afema®).

Pharmaceutical compositions suitable for use in the present disclosure include compositions wherein the antiestrogen is contained in a therapeutically or prophylactically effective amount, i.e., in an amount effective to achieve therapeutic or prophylactic benefit, as previously discussed. Of course, the actual amount effective for a particular application will depend, among other things, on the condition being treated and the route of administration. Determination of an effective amount is well within the capabilities of those skilled in the art, especially in light of the disclosure herein.

Patient doses for oral administration of the antiestrogen compound typically range from about 0.1 mg to about 100 mg/day or every other day, depending on the drug, the weight of the patient, and the severity of the symptoms. The dosage may be administered once every other day, once per day, or several or multiple times per day. The amount of the antiestrogen compound administered to practice methods of the present disclosure will, of course, be dependent on the subject being treated, the severity of the affliction, manner of administration and the judgment of the prescribing physician. The dose used to practice the disclosure can produce the desired therapeutic or prophylactic effects, without producing serious side effects.

In certain embodiments, a descending dosing schedule is employed. In one aspect, the descending dosing schedule comprises a daily descending dosing schedule. In one example of a daily descending dosing schedule, the initial dose of the antiestrogen is administered on day one, in one dose, or in two or more divided doses. On day two, a reduced dose of the antiestrogen is administered wherein the dose is reduced by 10% to 50% compared to the dose administered on day one. The reduced dose on day two may be administered in one dose, or in two or more divided doses. On day three, the antiestrogen is administered in a dose that is reduced by 10% to 50% of the dose administered on day two. The reduced dose on day three may be administered in one dose, or in two or more divided doses. The dosing schedule may be reduced on a daily basis until the dose on day n is 10% to 25% of the initial dose on day one. Optionally, the dose on day n may be repeated daily or on alternate days for up to two to four weeks. Two examples of daily descending dosing schedules can be found in Example 3. In one example, Subject C took single daily doses of Arimidex®: 1.0 mg on day one; 0.5 mg on day two; 0.25 mg each on days three and four. In another example, Subject C was administered a descending daily dosing schedule of tamoxifen citrate: 20 mg on day one; 10 mg on day two, 5 mg on day three and 2.5 mg each on days four and five. Subject C's last dose was 2.5 mg on the fifth day.

In another aspect, the descending dosing schedule may comprise a stepwise descending dosing schedule wherein the dose of an antiestrogen is held constant for two or more days at the initial dose, followed by a stepwise reduced dose, by 10% to 50% at each step. At each step the dose is held constant for two or more days, followed by a further 10% to 50% reduced dose step which is held constant for two or more days. The stepwise descending dosing schedule, wherein each step is held at a constant dose for two to seven day basis, is performed until the dose on day n is 10% to 25% of the initial dose on day one. Optionally, the dose on day n may be repeated daily or on alternate days for up to two to four weeks. Other descending dosing schedules are also possible and considered within the scope of the disclosure.

In one embodiment for the treatment of depression with an antiestrogen, a daily descending dosing schedule is employed. In one aspect, the antiestrogen is tamoxifen citrate and the dosing schedule comprises administering, for example, 20 mg base on day one, 10 mg on day two, 5 mg on day three, 2.5 mg on day four and 2.5 mg on day five. In another aspect, the antiestrogen is anastrozole and the dosing schedule comprises administering, for example, 1 mg on day one, 0.5 mg on day two, 0.25 mg on day three and 0.25 mg on day four. In another aspect, 0.25 mg anastrozole is further administered on day six and day eight.

In another embodiment for the treatment of bipolar disorder with an antiestrogen in an amount effective to alleviate mood swings, a once a day or alternate day dosing schedule is employed. In one aspect, the antiestrogen is tamoxifen citrate administered in the amount of about 10 to about 20 mg base taken regularly, generally once every day or once every other day. In another aspect, the antiestrogen is anastrozole which is administered in the amount of about 0.25 to about 1 mg taken regularly, generally once every day or once every other day.

In a further embodiment, the disclosure provides a method for the treatment of bipolar disorder comprising administration of an antiestrogen. In one aspect, qualified patients have been diagnosed with a bipolar illness by a certified psychiatrist prior to being considered as a candidate for treatment. The physician should perform a mental status exam on the patient, as to how they are presenting in the office at the time the antiestrogen is being considered. In an accompanying aspect, it is helpful to have a mood chart(s) done for some time before starting the new medication for the sake of comparison. The mood chart is used to ascertain mood problems, including time pattern and severity, and any accompanying symptoms (e.g. sleep status, psychotic symptoms, risk taking behavior, etc.).

In one aspect, prior to the beginning of treatment with an antiestrogen, all qualified patients (diagnosed bipolar by certified psychiatrist) should begin use of a mood chart, such as Gary Sach's Mood Chart, and continue use of the mood chart during the course of treatment to maintain a daily reference so that the doctor can better monitor their progress. In a preferred aspect, the patient fills out the mood chart on a regular basis and a family member, friend, roommate or clinical or institutional staff member who interacts with the patient on a regular basis, also records a parallel mood chart on a regular basis. This serves as a secondary reference to better help the treating physician make informed decisions. In another aspect, a mood chart is started at the start of treatment with an antiestrogen.

In one aspect, the antiestrogen is tamoxifen, or a pharmaceutical salt thereof. In a preferred aspect, the tamoxifen is in the form of tamoxifen citrate. Tamoxifen citrate is available from several generic drug manufacturers in either 10 mg or 20 mg tamoxifen base equivalent oral tablets. In another aspect, the tamoxifen citrate starting dose should be 10 mg to 20 mg base equivalents once every day for women, and 20 mg base equivalents once every day for men, with no change in the current medication schedule for other drugs during the first week. In a further aspect, starting in the second week of treatment, the dose of one or more of other current drugs taken to control bipolar symptoms is decreased by 10% to 25% while maintaining the tamoxifen citrate dose. In this aspect, a qualified doctor should determine which other current drugs should be decreased in dose and by how much. In another aspect, as positive results continue, the dosage of tamoxifen citrate can be decreased to down to 5 mg or 10 mg base equivalents once every day, or once every other day, as needed for maintenance of results. In one aspect, it is at the doctor's discretion how rapidly, or whether to continue to lower and/or eventually eliminate all other bipolar medications. This will vary patient by patient. Maintenance of the mood chart throughout the dosing period is performed to aid the doctor with evaluation of results. This dosing regimen results in decreased severity and/or decreased duration of episodes.

In another aspect, the antiestrogen is anastrozole. Anastrozole is available in 1 mg oral tablets as a generic drug from several manufacturers. In one aspect, the anastrozole starting dose should be 0.5 to 1 mg once every day, with no change in the current medication schedule for other drugs during the first week. In one aspect, starting in the second week after starting treatment with anastrozole, the dose of one or more of other current drugs taken to control bipolar symptoms should be decreased by 10% to 25% while maintaining the anastrozole. In this aspect, qualified doctor should determine which other current drugs should be decreased in dose and by how much. In another aspect, as positive results continue, the dosage of anastrozole can be decreased to down to 0.25 mg or 0.5 mg once every day or once every other day as needed for maintenance of results. In one aspect, it is at the doctor's discretion how rapidly, or whether to continue to lower and/or eventually eliminate all other bipolar medications. This will vary patient by patient. Maintenance of the mood chart throughout the dosing period is performed to aid the doctor with evaluation of results. This dosing regimen results in decreased severity and/or decreased duration of episodes.

### EXAMPLES

### Example 1

### Use of an Antiestrogen to Treat Symptoms of Bipolar Disorder in Subject A

Subject A is a 29 year old male in general good physical health with a family history of bipolar disorder. At ten years of age Subject A was diagnosed with ADHD and possible bipolar disorder. Imipramine hydrochloride (Tofranil®), a tricyclic antidepressant, 10 mg daily was prescribed to Subject A, who continued to have bipolar swings. Imipramine was later increased it to 25 mg daily, however Subject A had extreme hand tremors and found it difficult to write or draw. Subject A remained on this protocol for approximately 14-18 months.

Due to worsening side effects, imipramine was discontinued and Subject A was prescribed bupropion hydrochloride oral tablet (Wellbutrin®), which is used to treat the symptoms of depression, ADD and also bipolar. By the age of twelve Subject A was taking 250 mg of Wellbutrin twice daily. He continued this regimen until the age of fifteen, at which time Subject A made the personal decision to quit taking Wellbutrin, feeling that it dulled his mind and slowed his thinking.

Subject A took no medications for about five years. However, symptoms of bipolar disorder worsened until Subject A dropped out of college. Unable to hold a steady job, Subject A was formally diagnosed with bipolar disorder I by a psychiatrist. Following the diagnosis, Subject A was prescribed Eskalith® (Lithium Carbonate), a classic mood stabilizer and the most common bipolar drug due to its long term history and fair results. Also, due to a rare skin condition, Subject A was prescribed Dapsone to treat linear IgA dermatosis.

From beginning of treatment, no results were seen until the dosage of Eskalith was increased to 525 mg twice a day. Dosage was eventually 675 mg twice a day. This protocol slowed the swings, but did not change the severity of either the depressive or manic episodes. Also, due to damage to the thyroid, Synthroid® (levothyroxine sodium) 100 mcg/day was added.

Due to continued problems, other drugs were combined with Eskalith® in an attempt to stabilize Subject A's mood swings. The following list describes the drugs that were tried in combination with Eskalith:
1. Depakote® (Divalproex Sodium Delayed Release Tablets) 50 mg daily. This was discontinued due to heavy side effects.
2. Tegretol XR® (Carbamazepine) 200 mg twice daily. Worked for a few weeks and again discontinued due to hand and body tremors, as well as slurring of speech and numb feeling tongue.
3. Seroquel® (quetiapine fumarate) 25 mg twice daily. Discontinued due to extreme tiredness and inability to function.
4. Risperdal® (Risperidone) 3 mg per day. Discontinued due to inability to work or function to a satisfactory level.
5. Prozac® (Olanzapine and Fluoxetine hydrochloride) 20 mg twice daily. Discontinued due to increase in mania.
6. Klonopin® (Clonazepam): originally from 2002 to 2009 was 8 mg to 10 mg daily for generalized anxiety. Subject A currently continues taking 1-2 mg/day.
7. Lamictal® (Lamotrigine) Titration schedule over five weeks up to 250 mg twice daily.

Due to medical complications, (worsening skin condition; aggravated linear IgA dermatosis) Lamictal was discontinued and Eskalith was taken alone. Mood swings were more frequent without Lamictal.

Prednisone was prescribed for both the Linear IgA dermatosis and IBS syndrome. Prednisone was immediately discontinued as it made all physical and mental conditions worse.

In desperation, Subject A, tried taking 200 mg/week of Nandrolone deconate, hoping the anti-inflammatory aspects of the drug would help with physical conditions. This was done without a doctor's supervision. The mixture of high altitude and increased androgens caused Erythrocytosis (raised EPO production=>increased mass of red blood cells) in Subject A; forcing Phlebotomy and cessation of the Anabolic Steroid.

After the Erythrocytosis was brought under control, Subject A's regular testosterone production was low and the symptoms of bipolar disorder were still worsening. Subject A began to use the antiestrogen Arimidex® in an attempt to control rising estrogen. Within a week of starting Arimidex at 1 mg once a day, Subject A noticed the both episodes and symptoms became less severe. Subject A also that the cycles were less frequent while taking the antiestrogen. Subject A started self-discontinuing the Eskalith prescribed to treat his bipolar disorder.

Within a few months, Subject A had discontinued the Eskalith and found that taking only the Arimidex® daily not only returned his endocrine system to normal, but also substantially alleviated symptoms of his bipolar disorder. After taking Arimidex 1 mg once a day for 6 months, Subject A switched to tamoxifen citrate 20 mg once a day for several months without a change in stability.

For the previous 21 months to the present, Subject A has been without a bipolar swing while taking only an antiestrogen. Subject A surprisingly found that use of either Arimidex® or Tamoxifen citrate controls mood swings without the concurrent use of other bipolar medication.

Since beginning treatment using an antiestrogen, Subject A has remained stable for 19 months without incident. During the early portion of treatment, Subject A took tamoxifen citrate, 20 mg base equivalents, once every day; and later switched to Arimidex, 1 mg, once every day. Subject A's mood swings became virtually non-existent with his only bipolar (mood-stabilizing) medication remaining either tamoxifen citrate, 20 mg base equivalents, or 1 mg of Arimidex taken regularly; generally once every day or once every other day. Subject A now lives a normal life, maintains a successful business and marriage while also attending school to finish his degree. All family members state that Subject A is a completely different person, finally meeting his potential. They further state he is no longer violent, delusional, paranoid but remains gentle and understanding. Subject A no longer has problems with extreme depression (i.e. suicidal thoughts, catatonic episodes, inability to function) or mania (i.e. anger, violence, unreasonable spending, life-threatening actions, drug abuse). Subject A is currently taking Klonopin®, 2 to 4 mg daily, Levothyroxin, 100 mcg once a day, and Tamoxifen citrate, 20 mg once every other day, as needed.

Use of an antiestrogen, as a single medication to treat the symptoms of bipolar disorder in Subject A, was found to be far more effective than use of other bipolar medications, or combinations thereof, currently on the market. Not only does the antiestrogen work alone, but its use diminishes both the severity and frequency of a bipolar swing

### Example 2

### Use of an Antiestrogen to Treat Symptoms of Bipolar Disorder in Subject B

Subject B is a 56 year old female in good general physical health with a family history of bipolar disorder. At the age of 26, Subject B underwent Cholecystectomy. At the age of 37, Subject B was diagnosed with Meniere's Syndrome. At the age of 54, Subject B was diagnosed with bipolar disorder II, at which time Lamictal was prescribed by a psychiatrist. Lamictal was taken for ten to twelve weeks and discontinued due to memory loss and black outs. The psychiatrist then prescribed Effexor XR® (venlafaxine hydrochloride) 75 mg/daily. This was discontinued after three days due to lack of sleep, extreme mania, high anxiety and hyperactivity. A general physician prescribed 1.5 mg/Klonopin® (Clonazepam) daily.

In December 2008, Subject B started taking tamoxifen citrate or Arimidex® daily. For two weeks Subject B took 20 mg base equivalents of tamoxifen citrate daily, and gradually noticed increased mood stability and overall feeling of well being. Subject B also remained mentally alert and stable. Stress and any incidents that caused stress or anger were found to be short-lived and non-consequential. Due to increased mood stability, Subject B decreased the dosage to 10 mg of tamoxifen citrate every other day. When tamoxifen citrate was unavailable, Arimidex 0.25-1 mg was used interchangeably, without change in response. Currently, Subject B remains stable and emotionally level. Subject B has tried Arimidex and tamoxifen citrate and has been without incident.

Subject B has remained stable since beginning her treatment with tamoxifen citrate (approximately 18 months). Subject B no longer has bipolar symptoms with her only bipolar (mood-stabilizing) medication remaining tamoxifen citrate 10 mg or 20 mg base equivalents taken regularly. Subject B also found Arimidex to work equally as well, although the price of the medication is prohibitive. Subject B's husband states that Subject B is much more level, enjoys her life to a new degree, no longer loses control or becomes violent without warning. The entire family feels that life is much easier since Subject B began taking the antiestrogen.

The antiestrogen, as a single medication, is far more effective than any bipolar medication currently on the market. Not only does it work alone, but diminishes the severity and frequency of a bipolar swing. Subject is currently taking Klonopin® 1.5 to 2 mg daily and tamoxifen citrate 10 mg every other day. Subject B also is prescribed HCTZ (Hydrochlorothiazide) 50 mg as needed for symptoms of Meniere's Syndrome.

### Example 3

### Use of an Antiestrogen to Treat Symptoms of Acute/Severe Depression in Subject C

Subject C is a 26 year old female in good general physical health with no previous history of mental illness or depression. Subject C was prescribed Dapsone as a child in order to treat to treat dermititis herpetiformis. Subject C received no medication in the past four years. In December 2008, Subject C became lethargic, uninterested in usually pleasurable activities, restless, and began oversleeping. After a full month, symptoms of depression became extreme and subject C was thought to be in danger of becoming suicidal.

Subject C received a single dose of Tamoxifen citrate, 20 mg, on day one, and symptoms of depression lessened within the first eight hours. A descending daily dosing schedule was employed with administration of tamoxifen citrate: 10 mg on day two, 5 mg on day three and 2.5 mg each on day four and day five. Subject C's last dose was 2.5 mg on the fifth day. Symptoms of depression were alleviated and Subject C returned to normal activities, including law school.

After three months with no symptoms of depression, Subject C's acute depression recurred. Suicidal thoughts returned and Subject C considered dropping out of law school. Subject C started a descending dose course of Arimidex®; starting with a single dose of 1 mg on day one. Again, symptoms of depression faded noticeably within 8 hours, in a manner similar to that in response to treatment with tamoxifen. Subject C took single daily doses of Arimidex®; 0.5 mg on day two; 0.25 mg each on days three and four. Symptoms of depression were substantially alleviated after day four. After five months, Subject C remains free of symptoms of depression.

Use of an antiestrogen instead of a typical anti-depressant was found to be effective at alleviating symptoms of depression, creating a quick and stable recovery in 3-4 days. This may point to causes other than neurological for depression.

### Example 4

### Use of an Antiestrogen to Treat Symptoms of Bipolar Disorder in Subject D

Subject D is an 18 year old male with a family history of bipolar disorder. Although Subject D had symptoms spanning Asperger's Syndrome, Bipolar I, Obsessive-Compulsive Disorder, Oppositional Defiant Disorder and anti-social traits, bipolar I was the diagnosis for several reasons, including the fact that it was present on both sides of the family and the symptoms were better suited to a bipolar diagnosis.

Subject D's mother indicated that pregnancy was normal with no use of drugs, smoking or alcohol. Subject D's development was average or above, walking at 1, precocious, with a normal activity level. He has several sensory disorders, hypersensitivity to sounds, tactile defensiveness and inconsistent sleeping. At 4½ he was evaluated and found to be bright but to have traits that would resemble ADHD, Generalized Anxiety and Oppositional Defiant Disorder. A neuropsychological evaluation was provided from the time Subject D was twelve years old. Subject D has a history of extreme emotional rages. Unclear of the specific date, Subject D was placed on the SSRI Luvox 100 mg qam and qhs. He had, and continues to have, an anorexic preoccupation. Subject D has asthma, does not drink or smoke and is near sighted. He has had numerous bad experiences with bipolar and was first hospitalized at the age of 13 or 14. Prior to entering the study, Subject D was not doing well in college, was hospitalized at the beginning of the year prior to entering the study and had changed medications.

First visit: Day 1. Due to Subject D's extreme condition, past hospitalizations and the complicated mixture of medications, the psychiatrist spent approximately one hour with the subject. During this time, Subject D was informed about the possible side effects of tamoxifen citrate. Subject D signed the Informed Consent Form, and was given a supply of tamoxifen citrate. Subject D was also given a copy of the Gary Sach's Mood Chart for the subject and his mother to be filled out daily for the psychiatrist's weekly review.

Medications as of Day 1:

| Current Medications: | Change in Dosage: |
|---|---|
| Lithium ER 1350 mg qd | Tamoxifen citrate 20 mg qd |
| Depakote ER 1500 mg qd | added |
| Risperidone 4 mg qd | |
| Geodon 160 mg qd | |
| Vistaril (Vistaril?)prn | |
| DDAVP 0.01% Qh | |

Second visit: Day 9: Subject D's mother noticed and felt that the subject was far better. Subject D was concerned about sleeping too much. Subject D stated to the study coordinator that he began feeling more balanced after only four days on the tamoxifen citrate. Subject D told the psychiatrist that he was happier than he has been in a long time and has more energy.

Medications as of Day 9:

| Current Medications: | Change in Dosage: |
|---|---|
| Tamoxifen citrate 20 mg qd | Depakote reduced to 1000 mg qd |
| Lithium ER 1350 mg qd | Lithium ER 900 mg am/450 pm |
| Depakote ER 1500 mg qd | Risperidone 2 mg qhs |
| Risperidone 4 mg qd | |
| Geodon 160 mg qd | |
| Vistaril prn | |
| DDAVP 0.01% Qhs | |
| Doxycycline 100 qd | |

Third visit: Day 23: Subject D appeared to have forgotten the Lithium ER 450 evening dose. Subject D stated that he was working out, had more energy and felt better than he had in some time. The study psychiatrist discussed the study protocol with Subject D's personal psychiatrist who was skeptical and related she would drop the subject if he stayed on the study. Subject D and his family determined they did not want to stop study participation, so his personal psychiatrist refused to see him further, instead making him see her nurse. Due to the potentially upsetting change in Subject D's routine, the study psychiatrist made no changes in medication and asked the subject to stay in touch with either him or the study coordinator. The study coordinator called the subject and found the subject to be calm, clear thinking and stable even with the dramatic events of that week.

Medications as of Day 23:

| Current Medications: | Change in Dosage: |
|---|---|
| Tamoxifen citrate 20 mg qd | Lithium ER 900 mg qd |
| Lithium ER 900 mg | No changes in dosage |
| Depakote ER 1000 mg qd | |
| Risperidone 2 mg qd | |
| Geodon 160 mg qd | |
| Vistaril prn | |
| DDAVP 0.01% Qhs | |
| Doxycycline 100 qd | |

Fourth visit: Day 32: Subject D stated that he had about two days where he was hyper and had not gotten to the gym. Although hyper, Subject D did not feel manic or have manic symptoms. After the previous week, Subject D continued to remain stable, stating that he had been feeling good and had not had a day when he was unable to function. Subject D's mother had some concern that the subject was slightly manic; however, stated that he was better than he had ever been. Subject D showed no manic symptoms and stated to study coordinator that he felt his mind was working better and although worried that he was becoming manic, he was showing no symptoms that usually accompanied his typical mania.

Medications as of Day 32:

| Current Medications: | Change in Dosage: |
|---|---|
| Tamoxifen citrate 20 mg qd | Tamoxifen citrate increased to |
| Lithium ER 900 mg am | 30 mg qd |
| Depakote ER 1000 mg qd | Lithium ER reduced to 600 mg qd |
| Risperidone 2 mg qd | Depakote ER reduced to 750 mg qd |
| Geodon 160 mg qd | |
| Vistaril prn | |
| DDAVP 0.01% Qhs | |
| Doxycycline 100 qd | |

Fifth visit: Day 46: Subject D stated that he was feeling consistently better. The psychiatrist did not decrease his Geodon since Subject D was planning to travel for approximately a month. Subject D stated that he was able to be social and spend time with friends. He was very personable to everyone in the office and showed no symptoms of bipolar. Subject D's mother said that the study was: “A god send, and the best thing that has ever happened to their family”. Subject was concerned about his acne and mentioned he has been on Accutane in the past. Due to Accutane's known side effect of depression, subject was going to closely monitor his moods.

Medications as of Day 46:

| Current Medications: | Change in Dosage: |
|---|---|
| Tamoxifen citrate 30 mg qd | Depakote ER reduced to 500 mg qd |
| Lithium ER 600 mg am | Lithium ER reduced mgto 450 mg qd |
| Depakote ER 750 mg qd | |
| Risperidone 2 mg qd | |
| Geodon 160 mg qd | |
| Vistaril prn | |
| DDAVP 0.01% Qhs | |
| Doxycycline 100 qd | |

On Day 92, Subject D called the study coordinator from out of town, and stated that his family can see a night and day difference in him, and was remaining more stable than any time in his life and that his family feels the same way. Subject D also noted that even his friends had noticed a large difference. Subject noted that he was continuing to work out and would see the psychiatrist the first week he was back in town.

Sixth visit: Day 95: Subject D continued to remain stable, noting that he was feeling emotions differently than in the past. Due to Subject D's problems with sleeping, Dr. Simon gave a sample of Ambien for trial while the subject was gone. Subject stated that the Ambien has helped his sleeping. He was sleeping well and has had no depression or mania. Subject said he was slightly irritable on one occasion with very moderate anxiety. Subject D was also put on Accutane 30 mg qd during his vacation, but still shows no signs of depression.

Medications as of Day 95:

| Current Medications: | Change in Dosage: |
|---|---|
| Tamoxifen citrate 30 mg qd | Reduced Geodon by 40 mg to 120 mg |
| Lithium ER 450 mg qd | Added Ambien 10 mg pohs |
| Depakote ER 500 mg qd | Added 30 mg Accutane qd |
| Risperidone 2 mg qd | |
| Geodon 160 mg qd | |
| Vistaril prn | |
| DDAVP 0.01% Qhs | |
| Accutane 30 mg qd | |

Seventh visit: Day 111: Subject D continued to remain stable. He hurt his right ankle on Day 97, but had no mood changes, no anxiety, irritability or elevated moods. Subject was doing extremely well even as he continued to lower his other medications. He remarked he would get his ankle examined and return in one week.

Medications as of Day 111:

| Current Medications: | Change in Dosage: |
|---|---|
| Tamoxifen citrate 30 mg qam | Lithium ER reduced to 300 mg qd |
| Lithium ER 450 mg qd | |
| Depakote ER 500 mg qd | |
| Risperidone 2 mg qd | |
| Geodon 120 mg qd | |
| Vistaril prn | |
| DDAVP 0.01% Qhs | |
| Accutane 30 mg qd | |

Eighth visit: Day 118: Subject D continued to remain stable with some mood elevation, however said he feels it was very minor and easier to control. He was sleeping fine with no anxiety or irritability. Subject stated that he would like to continue decreasing his medication. Subject D contacted the study coordinator and was pleased he was able to put in a deposit for massage school. Before the study, subject stated that the idea was overwhelming but now felt even and controlled and ready to resume a normal life.

Medications as of Day 118:

| Current Medications: | Change in Dosage: |
|---|---|
| Tamoxifen citrate 30 mg qd | Geodon lowered to 80 mg qd |
| Lithium ER 300 mg am | |
| Depakote ER 500 mg qd | |
| Risperidone 2 mg qd | |
| Geodon 120 mg qd | |
| Vistaril prn | |
| DDAVP 0.01% Qhs | |
| Accutane 30 mg qd | |

Ninth visit: Day 125: Due to cost of medication, Subject D lowered his Risperidone from 2 mg to 1 mg. The change in medication, however, did not change the subject's mood stability or bother the subject in any way. Patient was also out of Maxalt 10 mg for occasional migraines. Rx for Maxalt 10 mg #10 1 at onset was given.

Medications as of Day 125:

| Current Medications: | Change in Dosage: |
|---|---|
| Tamoxifen citrate 30 mg qd added | Risperidone 1 mg qd |
| Lithium ER 300 mg qd | Rx Maxalt, occasional use for |
| Depakote ER 500 mg qd | migraines |
| Risperidone 1 mg qd | Reduced Depakote ER to 250 mg |
| Geodon 80 mg qd | |
| Vistaril prn | |
| DDAVP 0.01% Qhs | |
| Accutane 30 mg qd | |

Tenth visit: Day 133: Subject D felt ready to continue decreasing his medications. Subject D discontinued the Lithium.

Medications as of Day 133:

| Current Medications: | Change in Dosage: |
|---|---|
| Tamoxifen Citrate 30 | Added Ambien 10 mg as needed |
| mg qd | for sleep |
| Lithium ER 300 mg qd | discontinued Lithium ER |
| Depakote ER 250 mg qd | |
| Risperidone 1 mg qd | |
| Geodon 80 mg qd | |
| DDAVP 0.01% Qhs | |
| Accutane 30 mg qd | |

Eleventh Visit: Day 153: Subject D is excited about starting school, stating that it was something he thought he would never be able to do. Medications will remain the same until next visit.

Medications as of Day 153:

| Current Medications: | Change in Dosage: |
|---|---|
| Tamoxifen Citrate 30 mg qd | No changes |
| Depakote ER 250 mg qd | |
| Risperidone 1 mg qd | |
| Geodon 80 mg qd | |
| DDAVP 0.01% Qhs | |
| Accutane 30 mg qd | |

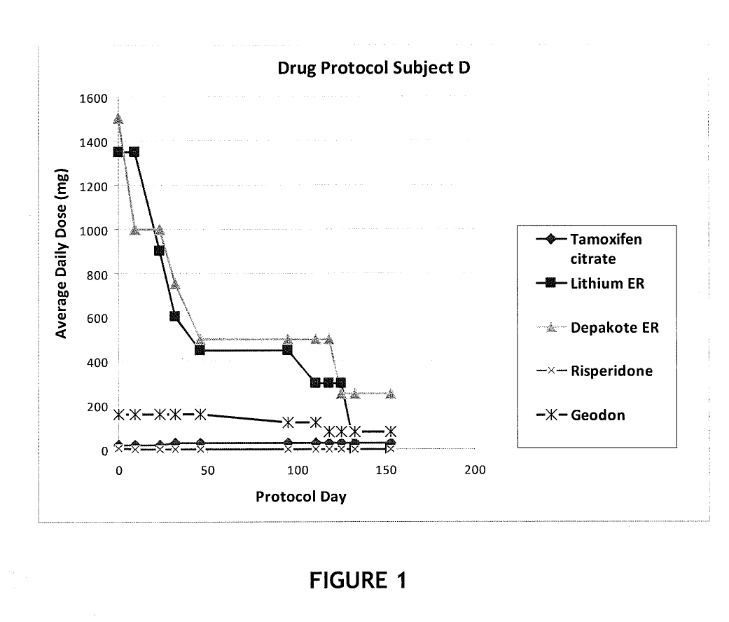

Synopsis: Subject D exhibited surprising improvement in daily functioning as reported by his family members, friends, relatives and the subject himself. Subject D continued participation in the pilot study and was doing well. For Subject D, with the use of a daily dose of 20-30 mg tamoxifen, the doses of Depakote ER, Risperidone and Geodon were each able to be significantly reduced and Lithium was able to be discontinued. The drug protocol is shown in Table 1 and in FIG. 1.

TABLE 1
Drug Protocol for Subject D

| | Drug (average daily dose, mg) | | | | |
|---|---|---|---|---|---|
| | Tamoxifen | Lithium | Depakote | | |
| Day | citrate | ER | ER | Risperidone | Geodon |
| 0 | 20 | 1350 | 1500 | 4 | 160 |
| 9 | 20 | 1350 | 1000 | 2 | 160 |
| 23 | 20 | 900 | 1000 | 2 | 160 |
| 32 | 30 | 600 | 750 | 2 | 160 |
| 46 | 30 | 450 | 500 | 2 | 160 |
| 95 | 30 | 450 | 500 | 2 | 120 |
| 111 | 30 | 300 | 500 | 2 | 120 |
| 118 | 30 | 300 | 500 | 2 | 80 |
| 125 | 30 | 300 | 250 | 1 | 80 |
| 133 | 30 | 0 | 250 | 1 | 80 |
| 153 | 30 | 0 | 250 | 1 | 80 |

Mood charts indicated that the subject did not suffer from swings of depression or mania and remained stable and balanced. Subject's changes exceeded expectations and are congruent with other participants within the pilot study. Subject felt stable enough to start massage therapy school. Before the study, the subject commented that the very idea of school and being around people was overwhelming, but he had no problems signing up for classes and dealing with the stress of starting school. Subject D noted that throughout the study he noticed a dramatic change in his attitude towards life, his own behavior and everyday tasks. He felt less cynical, more hopeful and under control.

### Example 5

### Use of an Antiestrogen to Treat Symptoms of Bipolar Disorder in Subject E

Subject E is a 47 year old male and an insulin dependent diabetic. Subject E was diagnosed with bipolar disorder at about the age of 38. He has had surgery in the past for his knees, his neck and stitches for his head. Subject E has been institutionalized multiple times within the last nine years for his own protection. To date, no medications have seemed to help. Subject also has a history of alcoholism. Some complexity was expected in treatment due to diabetes.

First Visit: Day 0: The psychiatrist spent an hour with Subject E in review of past medical history as well as medications and discussed the use of tamoxifen citrate with regard to bipolar with the subject's mother. Subject E signed the informed consent form and both he and his mother were sent home with the Gary Sach's mood chart and tamoxifen citrate.

Medication as of Day 0:

| Current Medications: | Change in Dosage: |
|---|---|
| Topamax 25 mg 2 tab hs | Added Tamoxifen citrate 20 mg qd |
| Fluoxetine 20 mg bid | |
| Trazadone HCl 150 mg hs | |
| Abilify 20 mg qd | |
| Doxylamine succinate 25 mg | |
| Lisinopril 5 mg qd | |
| Insulin Lantus 30 units | |
| Novalog 5-10 units before dinner | |
| Acid reducer | |
| Ranitidine 150 mg qd | |
| One a Day Tablet | |

Medications in italics not applicable to Bipolar Pilot Study and are not shown again.

Second Visit: Day 5: Subject E and his mother both stated he was doing extremely well. Subject was sleeping better but no longer staying in bed all day. Subject was coming down from his room and is more sociable than in the past and his attitude had improved. Subject E had missed a few doses of Abilify, so the psychiatrist gave the subject 10 mg samples while trying to lower it to a steady 5 mg qd.

Medication as of Day 5:

| Current Medications: | Change in Dosage: |
|---|---|
| Tamoxifen Citrate 20 mg qd | Fluoxetine reduced to 20 mg qd |
| Topamax 25 mg 2 tab hs | Abilify 10 mg with taper to 5 |
| Fluoxetine 20 mg bid | mg qd |
| Trazadone HCl 150 mg hs | |
| Abilify 20 mg qd *(Subject missed | |
| four days of this) | |
| Doxylamine succ 25 mg | |

Third Visit: Day 14: Subject E and his family continued to be pleased with the subject's improvements. Mostly, Subject E is showing a much calmer and sociable behavior. All aspects have either remained the same or improved. Subject needed more Abilify but could not afford it, so the psychiatrist is trying to get a few samples.

Medication as of Day 14:

| Current Medications: | Change in Dosage: |
|---|---|
| Tamoxifen Citrate 20 mg qd | No changes |
| Topamax 25 mg 2 tab hs | |
| Fluoxetine 20 mg qd | |
| Trazadone HCl 150 mg hs | |
| Abilify 5 mg qd | |
| Doxylamine succinate 25 mg | |

Fourth Visit: Day 27: Subject E could no longer get the Abilify through the Assistance Program, and since he could not afford it, he self-discontinued Abilify and experienced some depression and mild anxiety and irritability. Subject E was more depressed with mild anxiety.

Medication as of Day 27:

| Current Medications: | Change in Dosage: |
|---|---|
| Tamoxifen Citrate 20 mg qd | Abilify 5 mg (samples) |
| Topamax 25 mg qd | |
| Fluoxetine 20 mg qd | |
| Trazadone HCl 150 mg hs | |
| Abilify 5 mg qd (has not had for 7 days) | |
| Doxylamine succinate 25 mg | |

Fifth Visit: Day 34: Back on the Abilify the subject had a much better week. Considering the dosage cuts, Subject E responded well to the tamoxifen treatment. Subject stated that he felt more alert. Mother continued to see a large difference with the addition of the tamoxifen citrate 20 mg qd. Subject reported no irritability or anxiety in the last week.

Medication as of Day 34:

| Current Medications: | Change in Dosage: |
|---|---|
| Tamoxifen Citrate 20 mg qd | No changes |
| Topamax 25 mg qd | |
| Fluoxetine 20 mg qd | |
| Trazadone HCl 150 mg hs | |
| Abilify 5 mg qd | |
| Doxylamine succinate 25 mg | |

Sixth Visit: Day 56: Subject E's mother had been gone for two weeks, but on her return she stated to the psychiatrist that it was the best she had ever seen him. As a former gourmet chief, Subject E was well trained, but had not cooked since coming to live with his parents. Subject's mother stated that the subject was up on his own, and cooking all the meals. His mental state had improved dramatically and mother stated that it was the most sociable she had seen him. Diabetes was a concern for the psychiatrist and the Subject as his blood sugar ranged from 30-400, causing personal discomfort. Mental state remained steady and the Subject was cooperative and oriented. The psychiatrist noticed that the Abilify was taken occasionally at 7.5 mg qd. From the mother's and Subject's mood charts it looked like the 7.5 mg dosage was only taken two days. The psychiatrist reiterated that the dosage should remain at 5 mg qd.

Medication as of Day 56:

| Current Medications: | Change in Dosage: |
|---|---|
| Tamoxifen Citrate 20 mg qd | Abilify no more than 5 mg qd |
| Topamax 25 mg | |
| Fluoxetine 20 mg qd | |
| Trazadone Hcl 150 mg hs | |
| Ability 5-7.5 mg qd | |
| Doxylamine succinate 25 mg | |

Seventh Visit: Day 64: Subject remained mentally stable with the exception of one day of mild irritability with his mother. Mother stated that she instigated his irritability and his feelings were justified. Subject E, mother and the psychiatrist agreed it had been a good week and therefore continued lowering his other medications. The psychiatrist continued to observe that the diabetes was an issue and was causing health problems and would be monitored.

Medication as of Day 64:

| Current Medications: | Change in Dosage: |
|---|---|
| Tamoxifen citrate 20 mg qd | Fluoxetine lowered to 20 mg |
| Topamax 25 mg | every other day |
| Fluoxetine 20 mg qd | |
| Trazadone HCl 150 mg hs | |
| Abilify 5-7.5 mg qd | |
| Doxylamine succinate 25 mg | |

Eighth Visit: Day 72: Subject E had not suffered from anxiety in weeks, although the subject did have four days of depression; the mood charts filled out by both mother and subject denote a far less severe depression. Subject E and mother stated that his despondent behavior could have been due to the poor weather and cold [not necessarily caused by bipolar].

Medication as of Day 72:

| Current Medications: | Change in Dosage: |
|---|---|
| Tamoxifen citrate 20 mg qd | No change |
| Topamax 25 mg | |
| Fluoxetine 20 mg every other day | |
| Trazadone HCl 150 mg hs | |
| Abilify 5-7.5 mg qd | |

Ninth Visit: Day 85: Subject E expressed his happiness with how he is improving and feeling. Mother stated that the subject was doing better now than in the last ten years. Continued improvement was seen even as other drugs are being lowered.

Medication as of Day 85:

| Current Medications: | Change in Dosage: |
|---|---|
| Tamoxifen citrate 20 mg qd | Fluoxetine lowered to 20 mg |
| Topamax 25 mg | every 4^{th} day |
| Fluoxetine 20 mg every other day | |
| Trazadone HCl 150 mg hs | |
| Abilify 5-7.5 mg qd | |

Tenth Visit: Day 100: Subject E continued doing well and was stable despite receiving the news that his girlfriend was suffering from a terminal brain tumor.

Medication as Day 100:

| Current Medications: | Change in Dosage: |
|---|---|
| Tamoxifen Citrate 20 mg qd | No change |
| Topamax 25 mg qd | |
| Fluoxetine 10 mg qod (every other day) | |
| Trazadone HCl 150 mg hs | |
| Abilify 5 mg qd | |

Eleventh Visit: Day 106: Subject E came in with his father. He tried lowering the Abilify on his own, but felt that he needed the 2.5 mg

Medication as of Day 106:

| Current Medications: | Change in Dosage: |
|---|---|
| Tamoxifen Citrate 20 mg qd | No change |
| Topamax 25 mg qd | |
| Fluoxetine 10 mg qod (every other day) | |
| Trazadone HCl 150 mg hs | |
| Abilify 2.5 mg qd | |

Twelfth Visit: Day 113

Subject E woke up very confused. He took 3 glucose tables and was fine and although his blood sugar was not tested.

Medication as of Day 113:

| Current Medications: | Change in Dosage: |
|---|---|
| Tamoxifen Citrate 20 mg qd | No change |
| Topamax 25 mg qd | |
| Fluoxetine 10 mg qod (every other day) | |
| Trazadone Hcl 150 mg hs | |
| Abilify 2.5 mg qd | |

Thirteenth Visit: Day 120: Subject E continued to do well.

Medication as of Day 120:

| Current Medications: | Change in Dosage: |
|---|---|
| Tamoxifen Citrate 20 mg qd | Lowered Abilify to 2.0 mg |
| Topamax 25 mg qd | Lowered Topamax to 25 mg qod |
| Fluoxetine 10 mg qod (every other day) | |
| Trazadone HCl 150 mg hs | |
| Abilify 2.5 mg qd | |

Fourteenth Visit: Day 127: Subject E came in with his mood chart and reported a good week with no depression, anxiety, irritability or elevation of mood. He was doing well on the Abilify lowered to 2.0 mg Topamax was lowered to every third day.

Medication as of Day 127:

| Current Medications: | Change in Dosage: |
|---|---|
| Tamoxifen Citrate 20 mg qd | Lowered Topamax 25 mg to one |
| Topamax 25 mg qod | every three days |
| Fluoxetine 10 mg qod (every other day) | |
| Trazadone HCl 150 mg hs | |
| Abilify 2 mg qd | |

Fifteenth Visit: Day 141: Subject E had not exhibited any irritability or anxiety. Subject's mother states he has never been as good as in the last few months. Subject felt some depression but felt it is because he is sad about his girlfriend's brain tumor. Topamax and Fluoxetine were discontinued.

Medication as of Day 141:

| Current Medications: | Change in Dosage: |
|---|---|
| Tamoxifen Citrate 20 mg qd | dc (discontinued) Topamax |
| Topamax 25 mg every third day | dc (discontinued) Fluoxetine |
| Fluoxetine 10 mg qod (every other day) | |
| Trazadone HCl 150 mg hs | |
| Abilify 2 mg qd | |

Sixteenth Visit: Day 148: Due to blood sugar complication, Subject E was taken to the hospital where his blood sugar was 40 after having an IV. Despite his complications with diabetes, Subject E was doing extremely well. He was not depressed, manic or irritable. His mother stated that the subject was doing better all around than ever before.

Medication as of Day 148:

| Current Medications: | Change in Dosage: |
|---|---|
| Tamoxifen Citrate 20 mg qd | Abilify lowered to 1 mg qd |
| Trazadone HCl 150 mg hs | |
| Abilify 2 mg qd | |

Synopsis: Subject E exhibited significant improvement in daily functioning as reported by both his family members and the subject. Subject E continued participation in the pilot study. Subject's changes exceeded expectations and were congruent with other participants within the pilot study. Subject E is remained stable even though there is a great deal of pressure with his girlfriend's illness. He continued to attend AA meetings. Furthermore, Subject E felt that his diabetes was under control. His mood charts indicated continued progress and other medications will be continued to be lowered.

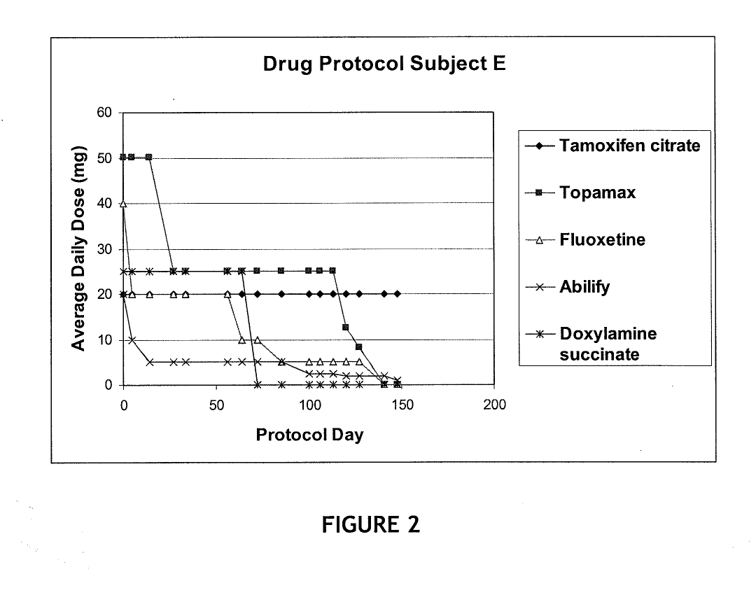

For Subject E, with the use of a daily dose of 20 mg tamoxifen, Topamax and Fluoxetine were able to be discontinued, and the dose of Abilify was significantly reduced. The drug protocol is shown in Table 2 and in FIG. 2.

TABLE 2
Drug Protocol for Subject E

| | Drug (average daily dose, mg)* | | | | |
|---|---|---|---|---|---|
| | Tamoxifen | | | | Doxylamine |
| Day | citrate | Topamax | Fluoxetine | Abilify | succinate |
| 0 | 20 | 50 | 40 | 20 | 25 |
| 5 | 20 | 50 | 20 | 10 | 25 |
| 14 | 20 | 50 | 20 | 5 | 25 |
| 27 | 20 | 25 | 20 | 5 | 25 |
| 34 | 20 | 25 | 20 | 5 | 25 |
| 56 | 20 | 25 | 20 | 5 | 25 |
| 64 | 20 | 25 | 10 | 5 | 25 |
| 72 | 20 | 25 | 10 | 5 | 0 |
| 85 | 20 | 25 | 5 | 5 | 0 |
| 100 | 20 | 25 | 5 | 2.5 | 0 |
| 106 | 20 | 25 | 5 | 2.5 | 0 |
| 113 | 20 | 25 | 5 | 2.5 | 0 |
| 120 | 20 | 12.5 | 5 | 2 | 0 |
| 127 | 20 | 8.3 | 5 | 2 | 0 |
| 141 | 20 | 0 | 0 | 2 | 0 |
| 148 | 20 | 0 | 0 | 1 | 0 |

*Note:
Trazadone HCl held constant at 150 mg hs throughout; not shown due to scale. See Example 5; medication at Day 0 for other drugs.

### Example 6

### Use of an Antiestrogen to Treat Symptoms of Bipolar Disorder in Subject F

Subject F is a 56 year old male who experienced a coronary three years prior to starting the study that his doctors blamed on stimulants used for ADD. Subject F had stents placed following the coronary. Subject had a long history of depression and ADD and was diagnosed with bipolar approximately four years ago by a trained psychiatrist. He maintains a job where he can do much of the work from home. It has been over a year since he was so severe he could not get out of bed or work. He has not had any extreme episodes in the immediate past. Subject F is nearsighted and has high blood pressure.

First Visit: Day 0: The psychiatrist thoroughly discussed the use of Tamoxifen citrate with regards to bipolar with Subject F, and spent an hour with patient in review of past medical history as well as medications. Subject F signed the informed consent form and both he and his spouse were sent home with the Gary Sach's mood chart and tamoxifen citrate.

Medication as of Day 0:

| Current Medications: | Change in Dosage: |
|---|---|
| Strattera 40 mg daily (has not been taking) | Added Tamoxifen citrate 20 |
| Adderal XR (has not been taking) | mg qd |
| Lamictal 200 mg qd | |
| Vyvanse | |
| Lisinopril qd | |
| Metoprolol bid | |
| Plavix 75 mg qd | |
| Fish Oil Capsules 1000 to 3000 mg daily | |

Medications in italics not applicable to Bipolar Pilot Study and are not shown again.

Second Visit: Day 13: Subject F had not been able to afford Strattera, stating that he had: “ . . . a lot of anger when not taking the Strattera.” Subject noted that with the addition of the tamoxifen he felt less angry even without taking the Strattera. Subject spoke with the study coordinator and stated that he had noticed a difference within three or four days of taking the tamoxifen citrate 20 mg qd. Subject had previously contacted study coordinator and stated that he had noticed a positive difference around four days after starting the Tamoxifen.

Medication as of Day 13:

| Current Medications: | Change in Dosage: |
|---|---|
| Tamoxifen citrate 20 mg qd | No changes |
| Strattera 40 mg daily (has not been taking) | |
| Adderal XR (has not been taking) | |
| Lamictal 200 mg qd | |
| Vyvanse | |

Third Visit: Day 28: The psychiatrist spoke with Subject F about filling out the mood charts and Subject stated that he now felt more comfortable filling out the charts and would begin as soon as possible. He was not having anger issues like he has had in the past, nor had any signs of depression or elevation of mood. Subject F had stopped taking Vyvanse.

Medication as of Day 28:

| Current Medications: | Change in Dosage: |
|---|---|
| Tamoxifen citrate 20 mg qd | Lamictal lowered to 100 |
| Strattera 40 mg daily (has not been taking) | mg qd |
| Adderal XR (has not been taking) | |
| Lamictal 200 mg qd | |
| Vyvanse | |

Fourth Visit: Day 41: The psychiatrist interviewed Subject F who stated that he could not notice a change with the lowered dosage of Lamictal. His wife had not complained about his irritability which is how he gauges if he is out of balance.

Medication as of Day 41:

| Current Medications: | Change in Dosage: |
|---|---|
| Tamoxifen citrate 20 mg qd | No changes |
| Strattera 40 mg daily (has not been taking) | |
| Adderal XR (has not been taking) | |
| Lamictal 100 mg qd | |

Subject F spoke with study coordinator on Day 52 and was very pleased with the results. Subject stated that he did not feel the tamoxifen like he does his other drugs, but is noticing the ability to sense his moods better and is overall extremely pleased and will continue the study.

Fifth Visit: Day 63: Subject F is doing well. He had been grouchy at night, but only to his wife. Comment was “But that may be necessary”. He did complete a mood chart and appeared more comfortable.

Medication as of Day 63:

| Current Medications: | Change in Dosage: |
|---|---|
| Tamoxifen citrate 20 mg qd | No changes |
| Strattera 40 mg daily (has not been taking) | |
| Adderal XR (has not been taking) | |
| Lamictal 100 mg qd | |

Sixth Visit: Day 70: Subject F had been taking the Tamoxifen since the end of April. He forgot it yesterday and thought he felt more depressed than any day since he started the program. He said worried about his weight gain since he stopped taking Vyvanse for his ADHD and feels that he may be putting things off more than previously due to this. He did not bring in his mood chart, however he did bring in his blood work.

Medication as of Day 70:

| Current Medications: | Change in Dosage: |
|---|---|
| Tamoxifen citrate 20 mg qd | Lamictal lowered to 50 mg qd |
| Strattera 40 mg daily (has not been taking) | Suggested Vitamin D |
| Adderal XR (has not been taking) | |
| Lamictal 100 mg qd | |

Seventh Visit: Day 77:

Subject E continues to do well and improve. He has been on the Tamoxifen since April. He continues to worry about his weight as it has increased since he went off the Vyvanse. He also stated that his job is not very demanding and may look for something else. He stated that one of the biggest problems in the past was the fact that he would spend money out of control and he has not had this problem since he went on the Tamoxifen. He has done well with the lowered dose of Lamictal and has seen no problem.

Medication as of Day 77:

| Current Medications: | Change in Dosage: |
|---|---|
| Tamoxifen citrate 20 mg qd | No changes |
| Strattera 40 mg daily (has not been taking) | |
| Adderal XR (has not been taking) | |
| Lamictal 50 mg qd | |

Eighth Visit: Day 84: Subject E had one outburst with his wife, the first since starting Tamoxifen. Overall, Subject E spoke with study coordinator and feels he is doing far better than he expected. He did feel like he is continuing to gain weight since stopping the Vyvanase. No change in his medication for visit.

Medication as of Day 84:

| Current Medications: | Change in Dosage: |
|---|---|
| Tamoxifen citrate 20 mg qd | No changes |
| Strattera 40 mg daily (has not been taking) | |
| Adderal XR (has not been taking) | |
| Lamictal 50 mg qd | |

Ninth Visit: Day 98: Subject E is not happy with his ADD since getting off of Vyvanase, but his moods remain steady. Dr. Simon discussed the risks of going back on Vyvanase due to his past heart problems. Lamictal was discontinued and Subject E is contemplating getting back on the Vyvanase despite the risks due to his ADD associated problems.

Medication as of Day 98:

| Current Medications: | Change in Dosage: |
|---|---|
| Tamoxifen citrate 20 mg qd | dc Lamictal since last visit |
| Strattera 40 mg daily (has not been | Will remain only on tamoxifen |
| taking) | for mood stabilization. |
| Adderal XR (has not been taking) | |
| Lamictal 50 mg qd | |

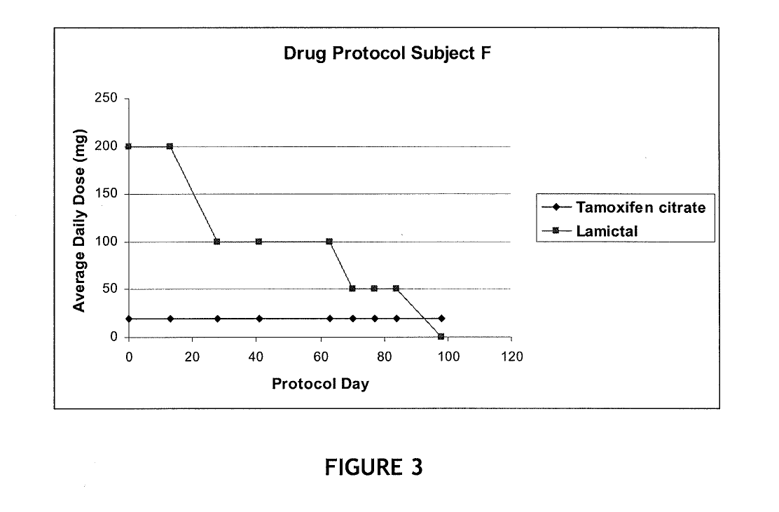

Synopsis. Patient has shown large improvement in daily functioning as reported by both his family members and the subject. Subject F is still continuing participation in the pilot study and doing well. Subject's changes met expectations and were congruent with other participants within the pilot study. Subject continued to show improvement in daily functioning. Subject felt that watching what he eats and exercising will help his weight and blood work. He will take the blood work to his cardiologist and we will discuss that and other changes at his next visit. Subject stated that since starting the Tamoxifen Citrate, his moods have been level, his normally “out of control spending” was no longer a problem and his anger and problems with wife have all but vanished. For Subject F, with the use of a daily dose of 20 mg tamoxifen, Lamictal was able to be discontinued, and Strattera was able to remain discontinued with mood stabilization. The drug protocol is shown in Table 3 and in FIG. 3.

TABLE 3
Drug Protocol for Subject F

Drug (average daily dose, mg)*

| Day | Tamoxifen citrate | Lamictal |
|---|---|---|
| 0 | 20 | 200 |
| 13 | 20 | 200 |
| 28 | 20 | 100 |
| 41 | 20 | 100 |
| 63 | 20 | 100 |
| 70 | 20 | 50 |
| 77 | 20 | 50 |
| 84 | 20 | 50 |
| 98 | 20 | 0 |

*See Example 6, Medication at Day 0 for other medication. Patient self-discontinued taking Straterra prior to study. Vyvanse/Adderal XR also were discontinued due to side effects.

The various embodiments described above are provided by way of illustration only and should not be construed to limit the disclosure. Those skilled in the art will readily recognize various modifications and changes that may be made to the present disclosure without following the example embodiments and applications illustrated and described herein, and without departing from the true spirit and scope of the present disclosure, which is set forth in the following claims.

1. A method of treating a bipolar disorder in a patient in need thereof, comprising administering a therapeutically effective amount of a pharmaceutically active antiestrogen, or a pharmaceutically acceptable salt thereof, in an amount effective to alleviate or prevent one or more symptoms of the bipolar disorder selected from the group consisting of mood swings and depressive mood episodes.

2. The method of claim 1 wherein the bipolar disorder is selected from the group consisting of bipolar disorder I, bipolar disorder II, cyclothymic disorder, and bipolar disorder not otherwise specified.

3. The method of claim 1 wherein the antiestrogen is selected from the group consisting of tamoxifen, raloxifene, anastrozole, letrozole, exemestane, vorozole, formestane, and fadrozole, or a pharmaceutically acceptable salt thereof.

4. The method of claim 3, wherein the antiestrogen is tamoxifen citrate.

5. The method of claim 4, wherein the tamoxifen citrate is administered in the amount of about 1 to 60 mg base equivalents once every day or once every other day.

6. The method of claim 5, wherein the tamoxifen citrate is administered in the amount of about 2.5 to about 40 mg base equivalents once every day or once every other day.

7. The method of claim 3, wherein the antiestrogen is anastrozole.

8. The method of claim 7, wherein the anastrozole is administered in the amount of about 0.25 to about 1 mg once every day or once every other day.

9. The method of claim 1, further comprising administering a pharmaceutically effective dose of at least one member of the group consisting of lithium, a pharmaceutical antidepressant, an anticonvulsant, a mood stabilizer, an antipsychotic agent and a benzodiazepine.

10. The method of treating a bipolar disorder of claim 1, wherein the therapeutically effective amount of an antiestrogen, or a pharmaceutically acceptable salt thereof,is an amount sufficient to reduce one or more of the severity of mood episodes and the frequency of depressive mood episodes in a subject in need thereof.

11. (canceled)

12. The method of claim 10 wherein the antiestrogen is selected from tamoxifen, anastrozole, or a pharmaceutically acceptable salt thereof.

13. The method of claim 12, wherein the pharmaceutically acceptable salt of tamoxifen is tamoxifen citrate.

14. A method of treating a clinical depression in a patient in need thereof, comprising administering a therapeutically effective amount of a pharmaceutically active antiestrogen, or a pharmaceutically acceptable salt thereof, in an amount effective to alleviate or prevent one or more symptoms of the depression.

15. The method of claim 14, wherein the depression is selected from unipolar depression, psychotic depression, major depressive disorder, dysthymic disorder, bipolar depression, treatment-resistant depression, single episodic and recurrent major depressive disorder.

16. The method of claim 15 wherein the bipolar depression is selected from the group consisting of bipolar disorder I, bipolar disorder II, and cyclothymic disorder.

17. The method of claim 14 wherein the antiestrogen is selected from one or more of the group consisting of tamoxifen, raloxifene, anastrozole, letrozole, exemestane, vorozole, formestane, and fadrozole, or a pharmaceutically acceptable salt thereof.

18. The method of claim 17, wherein the antiestrogen is tamoxifen citrate.

19. The method of claim 18, wherein the administering step comprises utilizing a descending dosing schedule of tamoxifen citrate.

20. The method of claim 19, wherein the descending dosing schedule of tamoxifen citrate comprises administering about 20 mg base equivalents on day one, about 10 mg base equivalents on day two, about 5 mg base equivalents on day three, about 2.5 mg base equivalents on day four and about 2.5 mg base equivalents on day five.

21. The method of claim 17, wherein the antiestrogen is anastrozole.

22. The method of claim 21, wherein the administering step comprises utilizing a descending dosing schedule of anastrozole.

23. The method of claim 22, wherein the descending dosing schedule of anastrozole comprises administering about 1 mg on day one, about 0.5 mg on day two, about 0.25 mg on day three and about 0.25 mg on day four.

24. The method of claim 23, further comprising administering about 0.25 mg of anastrozole on day six and about 0.25 mg on day eight.

25. The method of claim 14, wherein the administering step occurs at the first sign of an acute severe depression.

26. The method of claim 14, further comprising administering a pharmaceutically effective dose of at least one member of the group consisting of lithium, a pharmaceutical antidepressant, an anticonvulsant, a mood stabilizer, an antipsychotic agent and a benzodiazepine.

27. The method of claim 1, further comprising the steps of
observing stabilized behavior in the patient; and
decreasing the dose or discontinuing the administration of one or more other concurrently administered drugs selected from the group consisting of lithium, a pharmaceutical antidepressant, an anticonvulsant, a mood stabilizer, an antipsychotic agent and a benzodiazepine that is concurrently administered to the patient.

28. (canceled)
